(19) United States
(12) Patent Application Publication
(10) Pub. No.: US 2021/0401794 A1
(43) Pub. Date: Dec. 30, 2021

(54) Methods of Using Cannabinoid Compositions for Inducing and Promoting Sleep

(71) Applicant: NP Pharma Holdings, LLC, Sheridan, WY (US)

(72) Inventor: Peyton Palaio, Denver, CO (US)

(21) Appl. No.: 17/362,969

(22) Filed: Jun. 29, 2021

Related U.S. Application Data
(60) Provisional application No. 63/046,654, filed on Jun. 30, 2020.

(51) Int. Cl. A61K 31/352; A61K 31/165; A61K 31/4045; A61K 31/353; A61K 31/7048; A61K 36/28; A61K 31/164; A61K 31/366; A61K 31/16; A61K 31/683; A61K 31/198; A61K 31/231; A61K 31/557; A61K 31/08; A61K 31/232; A61K 31/05; A61K 45/06

(52) U.S. Cl. CPC A61K 31/352; A61K 45/06; A61K 31/4045; A61K 31/353; A61K 31/7048; A61K 36/28; A61K 31/164; A61K 31/366; A61K 31/16; A61K 31/683; A61K 31/198; A61K 31/231; A61K 31/557; A61K 31/08; A61K 31/232; A61K 31/05; A61K 31/165

(57) ABSTRACT

Provided are compositions comprising cannabinol; one or more agents selected from a flavanol, an endocannabinoid, capsaicin, dihydrocapsaicin, nordihydrocapsaicin, homocapsaicin, and homodihydrocapsaicin; and, optionally, melatonin, and methods of making such compositions. The disclosed compositions can be useful in, for example, treating a sleep disorder (e.g., insomnia, seasonal affective disorder, or jet lag). The disclosed compositions can also be useful in inducing and/or promoting sleep. This abstract is intended as a scanning tool for purposes of searching in the particular art and is not intended to be limiting of the present invention.

### CROSS-REFERENCE TO RELATED APPLICATIONS

This Application claims the benefit of U.S. application Ser. No. 63/046,654, filed on Jun. 30, 2020, the contents of which are hereby incorporated by reference in their entirety.

### BACKGROUND

Cannabinoids are a class of compounds that act on the cannabinoid receptors in cells. Cannabinoids can be endogenous (i.e., endocannabinoids; present endogenously in human or animal tissues), synthetic, or derived from plants (i.e., phytocannabinoids). A well-known source of phytocannabinoids is the genus of plants known as Cannabis or more colloquially referred to as marijuana. At least 104 phytocannabinoids have been isolated from marijuana to date, and many of these compounds have widely variable biological activities and properties. Cannabinoids have been reported to provide various therapeutic benefits for humans, including effects that are anti-psychotic, analgesic, anti-inflammatory, anti-spasmodic, anti-convulsant, anti-emetic, antioxidant, neuroprotective, and immunomodulatory. In addition, certain cannabinoids have been investigated for their benefit in sedation and in improving sleep; however, there are significant challenges and issues relating to their safe, effective, and federally acceptable use such as, for example, a wide range of side effects, a complex mechanisms of action, purity, consistency, quality control, product sourcing, and cost.

Thus, despite the potential benefits of using cannabinoids for inducing and promoiting sleep, the safe and effective incorporation of cannabinoids into sleep aide compositions has remained elusive. Thus, there remains a need for cannabinoid compositions and methods of making and using same. These needs and others are met by the present invention.

### SUMMARY

In accordance with the purpose(s) of the invention, as embodied and broadly described herein, the invention, in one aspect, relates to topical cannabinoid compositions and methods of making and using same in, for example, promoting and inducing sleep and/or treating sleep disorders (e.g., insomnia, seasonal affective disorder (SAD), and jet lag).

Thus, disclosed are compositions comprising: (a) cannabinol (CBN) in an amount of at least about 1 wt % based on the total weight of the composition; (b) one or more agents selected from a flavanol, an endocannabinoid, capsaicin, dihydrocapsaicin, nordihydrocapsaicin, homocapsaicin, and homodihydrocapsaicin; and (c) optionally, melatonin, wherein the composition is substantially free of surfactants.

Also disclosed are compositions comprising: (a) cannabinol (CBN) in an amount of at least about 1 wt % based on the total weight of the composition; and (b) melatonin, wherein the composition is substantially free of surfactants.

Also disclosed are compositions comprising: (a) cannabinol (CBN) in an amount of at least about 1 wt % based on the total weight of the composition; and (b) melatonin in an amount of from about 0.5 wt % to about 5 wt % based on the total weight of the composition, wherein the ratio of CBN to melatonin is of from about 1:1 by weight to about 5:1 by weight and wherein the composition is substantially free of surfactants.

Also disclosed are methods for making a disclosed composition.

Also disclosed are methods for promoting sleep in a subject, the method comprising administering to the subject an effective amount of a disclosed composition.

Also disclosed are kits comprising a disclosed composition, and one or more of: (a) a device selected from a vaporizer, a transdermal patch, or an inhaler; (b) an agent known to promote sleep and/or to treat a sleep disorder; (c) instructions for promoting sleep; and (d) instructions for treating a sleep disorder.

Still other objects and advantages of the present disclosure will become readily apparent by those skilled in the art from the following detailed description, wherein it is shown and described only the preferred aspects, simply by way of illustration of the best mode. As will be realized, the disclosure is capable of other and different aspects, and its several details are capable of modifications in various obvious respects, without departing from the disclosure. Accordingly, the description is to be regarded as illustrative in nature and not as restrictive.

### DETAILED DESCRIPTION

The present invention can be understood more readily by reference to the following detailed description of the invention and the Examples included therein.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present compositions, methods and systems may be understood more readily by reference to the following detailed description of preferred embodiments and the examples included therein and to the Figures and their previous and following description.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

Listed below are definitions of various terms used to describe this invention. These definitions apply to the terms as they are used throughout this specification, unless otherwise limited in specific instances, either individually or as part of a larger group.

As used in the specification and in the claims, the term “comprising” can include the aspects “consisting of” and “consisting essentially of.”

As used in the specification and the appended claims, the singular forms “a,” “an,” and “the” include plural referents unless the context clearly dictates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

“Optional” or “optionally” means that the subsequently described event or circumstance may or may not occur, and that the description includes instances where said event or circumstance occurs and instances where it does not.

As used herein, the term “by weight,” when used in conjunction with a component, unless specially stated to the contrary is based on the total weight of the formulation or composition in which the component is included. For example, if a particular element or component in a composition or article is said to have 8% by weight, it is understood that this percentage is in relation to a total compositional percentage of 100%.

A weight percent of a component, or weight %, or wt %, unless specifically stated to the contrary, is based on the total weight of the formulation or composition in which the component is included.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with an ailment, disease, or disorder. The term “patient” includes human and veterinary subjects.

As used herein, the term “treatment” refers to the medical management of a patient with the intent to cure, ameliorate, stabilize, or prevent an ailment, disease, pathological condition, disorder, or injury. This term includes active treatment, that is, treatment directed specifically toward the improvement of a disease, pathological condition, disorder, or injury, and also includes causal treatment, that is, treatment directed toward removal of the cause of the associated disease, pathological condition, disorder, or injury. In addition, this term includes palliative treatment, that is, treatment designed for the relief of symptoms rather than the curing of the disease, pathological condition, disorder, or injury; preventative treatment, that is, treatment directed to minimizing or partially or completely inhibiting the development of the associated disease, pathological condition, disorder, or injury; and supportive treatment, that is, treatment employed to supplement another specific therapy directed toward the improvement of the associated disease, pathological condition, disorder, or injury. In various aspects, the term covers any treatment of a subject, including a mammal (e.g., a human), and includes: (i) preventing the disorder or condition from occurring in a subject that can be predisposed to the disorder or condition but has not yet been diagnosed as having it; (ii) inhibiting the disorder or condition, i.e., arresting its development or exacerbation thereof; or (iii) relieving the disorder or condition, i.e., promoting healing of the disorder or condition. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compositions or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed composition or a product of a disclosed method of making, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed composition or a product of a disclosed method of making, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition) , and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-HIV agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro- drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

References in the specification and concluding claims to parts by weight of a particular element or component in a composition or article, denotes the weight relationship between the element or component and any other elements or components in the composition or article for which a part by weight is expressed. Thus, in a composition or a selected portion of a composition containing 2 parts by weight of component X and 5 parts by weight component Y, X and Y are present at a weight ratio of 2:5, and are present in such ratio regardless of whether additional components are contained in the composition.

As used herein, the term “substantially,” in, for example, the context “substantially free of” refers to a composition having less than about 10% by weight, e.g., less than about 5%, less than about 1%, less than about 0.5%, less than about 0.1%, less than about 0.05%, or less than about 0.01% by weight of the stated material, based on the total weight of the composition.

It is further understood that the term “substantially,” when used in reference to a composition, refers to at least about 60% by weight, e.g., at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by weight, based on the total weight of the composition, of a specified feature, component, or a combination of the components. It is further understood that if the composition comprises more than one component, the two or more components can be present in any ratio predetermined by one of ordinary skill in the art.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, and amides, salts of esters or amides, and N-oxides of a parent compound.

Disclosed are also components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

### B. COMPOSITIONS

In one aspect, disclosed are compositions comprising: (a) cannabinol (CBN) in an amount of at least about 1 wt % based on the total weight of the composition; (b) one or more agents selected from a flavanol, an endocannabinoid, capsaicin, dihydrocapsaicin, nordihydrocapsaicin, homocapsaicin, and homodihydrocapsaicin; and (c) optionally, melatonin, wherein the composition is substantially free of surfactants.

In one aspect, disclosed are compositions comprising: (a) CBN in an amount of at least about 1 wt % based on the total weight of the composition; and (b) melatonin, wherein the composition is substantially free of surfactants.

In one aspect, disclosed are compositions comprising: (a) CBN in an amount of at least about 1 wt % based on the total weight of the composition; and (b) melatonin in an amount of from about 0.5 wt % to about 5 wt % based on the total weight of the composition, wherein the ratio of CBN to melatonin is of from about 1:1 by weight to about 5:1 by weight and wherein the composition is substantially free of surfactants.

Thus, in various aspects, CBN is present in an amount of at least about 1 wt %, at least about 2 wt %, at least about 5 wt %, at least about 10 wt %, at least about 15 wt %, at least about 20 wt % at least about 25 wt %, at least about 30 wt %, at least about 35 wt %, or at least about 40 wt % based on the total weight of the composition.

In various aspects, the composition comprises one or more agents selected from a flavanol, an endocannabinoid, capsaicin, dihydrocapsaicin, nordihydrocapsaicin, homocapsaicin, and homodihydrocapsaicin. In a further aspect, the composition comprises a flavanol. In a still further aspect, the composition comprises an endocannabinoid. In yet a further aspect, the composition comprises one or more of capsaicin, dihydrocapsaicin, nordihydrocapsaicin, homocapsaicin, and homodihydrocapsaicin.

In various aspects, the composition comprises one or more agents in an amount of from about 0.5 wt % to about 30 wt %, about 0.5 wt % to about 25 wt %, about 0.5 wt % to about 20 wt %, about 0.5 wt % to about 15 wt %, about 0.5 wt % to about 10 wt %, about 0.5 wt % to about 8 wt %, about 0.5 wt % to about 6 wt %, about 0.5 wt % to about 4 wt %, about 0.5 wt % to about 2 wt %, about 0.5 wt % to about 1 wt %, about 0.5 wt % to about 30 wt %, about 1 wt % to about 30 wt %, about 2 wt % to about 30 wt %, about 4 wt % to about 30 wt %, about 6 wt % to about 30 wt %, about 8 wt % to about 30 wt %, about 10 wt % to about 30 wt %, about 15 wt % to about 30 wt %, about 20 wt % to about 30 wt %, about 25 wt % to about 30 wt %, about 1 wt % to about 25 wt %, about 2 wt % to about 20 wt %, about 4 wt % to about 20 wt %, about 6 wt % to about 15 wt %, or about 8 wt % to about 10 wt %, based on the total weight of the composition.

In various aspects, the flavanol is present. Examples of flavanols include, but are not limited to, kaempferol and rutin. In a further aspect, the flavanol is kaempferol. Thus, the flavanol can be present in an amount of from about 0.5 wt % to about 30 wt %, about 0.5 wt % to about 25 wt %, about 0.5 wt % to about 20 wt %, about 0.5 wt % to about 15 wt %, about 0.5 wt % to about 10 wt %, about 0.5 wt % to about 8 wt %, about 0.5 wt % to about 6 wt %, about 0.5 wt % to about 4 wt %, about 0.5 wt % to about 2 wt %, about 0.5 wt % to about 1 wt %, about 0.5 wt % to about 30 wt %, about 1 wt % to about 30 wt %, about 2 wt % to about 30 wt %, about 4 wt % to about 30 wt %, about 6 wt % to about 30 wt %, about 8 wt % to about 30 wt %, about 10 wt % to about 30 wt %, about 15 wt % to about 30 wt %, about 20 wt % to about 30 wt %, about 25 wt % to about 30 wt %, about 1 wt % to about 25 wt %, about 2 wt % to about 20 wt %, about 4 wt % to about 20 wt %, about 6 wt % to about 15 wt %, or about 8 wt % to about 10 wt %, based on the total weight of the composition.

In various aspects, the composition consists essentially of CBN, the flavanol, and melatonin. In a further aspect, the composition consists of CBN, the flavanol, and melatonin.

In various aspects, the endocannabinoid is present. Examples of endocannabinoids include, but are not limited to, 2arachidonoylglycerol (2-AG), 2-arachidonyl glyceryl ether (2-AGE), N-arachidonoyl dopamine (NADA), stearoylethanolamide (SEA), O-arachidonoyl ethanolamine (O-AEA), arachidonoyl ethanolamide (AEA), 2-oleoylglycerol (2-OG), N-arachidonylglycine (NAGly), 2-arachidonoyl lysophosphatidylinositol (2-ALPI), N-arachidonoyl serotonin (AA-5-HT), docosatetraenoylethanolamide (DEA), lysophosphatidylinositol (LPI), oleamide, oleoylethaolamide (OEA), palmitoylethanolamide (PEA), RVD-Hpa, stearoylethanolamide (SEA), yangonin, and an alkylamide (e.g., Echinacea and its constituents). Thus, the endocannabinoid can be present in an amount of from about 0.5 wt % to about 30 wt %, about 0.5 wt % to about 25 wt %, about 0.5 wt % to about 20 wt %, about 0.5 wt % to about 15 wt %, about 0.5 wt % to about 10 wt %, about 0.5 wt % to about 8 wt %, about 0.5 wt % to about 6 wt %, about 0.5 wt % to about 4 wt %, about 0.5 wt % to about 2 wt %, about 0.5 wt % to about 1 wt %, about 0.5 wt % to about 30 wt %, about 1 wt % to about 30 wt %, about 2 wt % to about 30 wt %, about 4 wt % to about 30 wt %, about 6 wt % to about 30 wt %, about 8 wt % to about 30 wt %, about 10 wt % to about 30 wt %, about 15 wt % to about 30 wt %, about 20 wt % to about 30 wt %, about 25 wt % to about 30 wt %, about 1 wt % to about 25 wt %, about 2 wt % to about 20 wt %, about 4 wt % to about 20 wt %, about 6 wt % to about 15 wt %, or about 8 wt % to about 10 wt %, based on the total weight of the composition.

In various aspects, the composition consists essentially of CBN, the endocannabinoid, and melatonin. In a further aspect, the composition consists of CBN, the endocannabinoid, and melatonin.

In various aspects, melatonin is present. Thus, melatonin can be present in an amount of from about 0.5 wt % to about 5 wt %, about 0.5 wt % to about 4 wt %, about 0.5 wt % to about 3 wt %, about 0.5 wt % to about 2 wt %, about 0.5 wt % to about 1 wt %, about 1 wt % to about 5 wt %, about 2 wt % to about 5 wt %, about 3 wt % to about 5 wt %, about 4 wt % to about 5 wt %, about 1 wt % to about 4 wt % or about 2 wt % to about 3 wt % based on the total weight of the composition.

In various aspects, the ratio of CBN to melatonin is of from about 1:1 by weight to about 5:1 by weight, from about 1:1 by weight to about 4:1 by weight, from about 1:1 by weight to about 3:1 by weight, from about 1:1 by weight to about 2:1 by weight, from about 2:1 by weight to about 5:1 by weight, from about 3:1 by weight to about 5:1 by weight, from about 4:1 by weight to about 5:1 by weight, or from about 2:1 by weight to about 4:1 by weight.

In various aspects, the composition consists essentially of CBN and melatonin. In a further aspect, the composition consists of CBN and melatonin.

In various aspects, the composition is substantially free of surfactants. Thus, in various further aspects, the composition contains less than about 10% by weight, less than about 5%, less than about 1%, less than about 0.5%, less than about 0.1%, less than about 0.05%, or less than about 0.01% by weight of surfactants based on the total weight of the composition.

In a further aspect, the composition further comprises an oil. Examples of oils include, but are not limited to, tocopheryl acetate, olive oil, almond oil, apricot kernel oil, avocado oil, borage seed oil, camellia seed oil (tea oil), cranberry seed oil, evening primrose oil, grapeseed oil, hazelnut oil, hemp seed oil, jojoba, kukui nut oil, macademia nut oil, meadowfoam oil, peanut oil, pecan oil, pomegranate seed oil, rose hip oil, seabuckthorn berry oil, sesame seed oil, coconut oil, sunflower oil, watermelon seed oil, and MCT oil, or mixtures thereof In a still further aspect, the oil is tocopheryl acetate.

Without wishing to be bound by theory, the amount of oil present can be dependent upon the formulation of the composition. For example, in various aspects, the oil is present in an amount of from about 20 wt % to about 30 wt %, 25 wt % to about 30 wt %, or 20 wt % to about 25 wt %. In various further aspects, the oil is present in an amount of from about 50 wt % to about 98 wt %, 60 wt % to about 98 wt %, 70 wt % to about 98 wt %, 80 wt % to about 98 wt %, 90 wt % to about 98 wt %, 50 wt % to about 90 wt %, 50 wt % to about 80 wt %, 50 wt % to about 70 wt %, or 50 wt % to about 60 wt %. In a further aspect, the oil is present in an amount of from about 50 wt % to about 98 wt % based on the total weight of the composition.

In various aspects, the oil is naturally occurring. In a still further aspect, the oil is non-naturally occurring (e.g., MCT oil).

In various aspects, the composition further comprises a cannabinoid selected from Δ9-tetrahydrocannabinol (THC), cannabidiol (CBD), cannabigerol (CBG), cannabicyclol (CBL), and cannabichromene (CBC), or a mixture thereof. Cannabinoids are a class of diverse chemical compounds that act on cannabinoid receptors on cells that repress neurotransmitter release in the brain. Cannabinoid receptors are of a class of cell membrane receptors under the G protein-coupled receptor superfamily. As is typical of G protein-coupled receptors, the cannabinoid receptors contain seven transmembrane spanning domains. There are currently two known subtypes of cannabinoid receptors, termed CB1 and CB2, with mounting evidence of more. The CB1 receptor is expressed mainly in the brain (central nervous system), but also in the lungs, liver and kidneys. The CB2 receptor is expressed mainly in the immune system and in hematopoietic cells.

The classical cannabinoids are derived from their respective 2-carboxylic acids (2-COOH) by decarboxylation, catalyzed by heat, light, or alkaline conditions. Phytocannabinoids (those derived from the Cannabis plant) include but not limited to: tetrahydrocannabinol (THC), tetrahydrocannabinolic acid (THCA), cannabidiolic acid (CBDA), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV) and cannabigerol monomethyl ether (CBGM). Thus, in various aspects, the cannabinoid is THC, CBD, THCA, CBDA, CBN, CBG, CBC, or CBGM, or a mixture thereof. In a further aspect, the cannabinoid is THC or CBD, or a mixture thereof. In a still further aspect, the cannabinoid is THC. In yet a further aspect, the cannabinoid is CBD.

In a further aspect, the cannabinoid is present in an amount of from about 1 wt % to about 20 wt % based on the total weight of the composition. Thus, in various aspects, the cannabinoid is present in an amount of from about 1 wt % to about 15 wt %, about 1 wt % to about 10 wt %, about 1 wt % to about 5 wt %, about 5 wt % to about 20 wt %, about 10 wt % to about 20 wt %, or about 15 wt % to about 20 wt % based on the total weight of the composition.

In various aspects, the composition further comprises a cannabinoid acid. Examples of cannabinoid acids include, but are not limited to, cannabigerolic acid (CBGA), Δ⁹-tetrahydrocannabinolic acid (THCA), cannabidiolic acid (CBDA), cannabichromenenic acid (CBCA), cannabigerovarinic acid (CBGVA), tetrahydrocanabivarinic acid (THCVA), cannabidivarinic acid (CBDVA), and cannabichromevarinic acid (CBCVA). In a further aspect, the cannabinoid acid is present in an amount of from about 1 wt % to about 15 wt %, about 1 wt % to about 10 wt %, about 1 wt % to about 5 wt %, about 5wt % to about 20 wt %, about 10 wt % to about 20 wt %, or about 15 wt % to about 20 wt % based on the total weight of the composition.

In various aspects, the composition is substantially free of THC. Thus, in various further aspects, the composition contains less than about 10% by weight, less than about 5%, less than about 1%, less than about 0.5%, less than about 0.1%, less than about 0.05%, or less than about 0.01% by weight of THC based on the total weight of the composition.

In various aspects, the composition is substantially free of CBN. Thus, in various further aspects, the composition contains less than about 10% by weight, less than about 5%, less than about 1%, less than about 0.5%, less than about 0.1%, less than about 0.05%, or less than about 0.01% by weight of CBN based on the total weight of the composition.

In various aspects, the composition is substantially free of THC and CBN. Thus, in various further aspects, the composition contains less than about 10% by weight, less than about 5%, less than about 1%, less than about 0.5%, less than about 0.1%, less than about 0.05%, or less than about 0.01% by weight of both THC and CBN based on the total weight of the composition.

In various aspects, the composition further comprises an additive. Examples of additives include, but are not limited to, diluents, buffers, binders, surface-active agents, lubricants, humectants, pH adjusting agents, preservatives (including anti-oxidants), emulsifiers, occlusive agents, opacifiers, antioxidants, colorants, flavoring agents, gelling agents, thickening agents, stabilizers, and surfactants, among others. Thus, in various further aspects, the additive is vitamin E, gum acacia, citric acid, stevia extract powder, Luo Han Gou, Monoammonium Glycyrhizinate, Ammonium Glycyrrhizinate, honey, or combinations thereof. In a still further aspect, the additive is a flavoring agent, a binder, a disintegrant, a bulking agent, and/or silica.

In a further aspect, the additive is present in an amount of from about 0.01 wt % to about 10 wt % based on the total weight of the composition. In a still further aspect, the additive is present in an amount of from about 0.01 wt % to about 8 wt %, from about 0.01 wt % to about 6 wt %, from about 0.01 wt % to about 4 wt %, from about 0.01 wt % to about 2 wt %, from about 0.01 wt % to about 1 wt %, from about 0.1 wt % to about 10 wt %, from about 1 wt % to about 10 wt %, from about 2 wt % to about 10 wt %, from about 4 wt % to about 10 wt %, from about 6 wt % to about 10 wt %, or from about 8 wt % to about 10 wt % based on the total weight of the composition.

In a further aspect, the additive is present in an amount of at least about 20 wt % based on the total weight of the composition. Thus, in various aspects, the additive is present in an amount of at least about 20 wt %, at least about 25 wt %, at least about 30 wt %, at least about 35 wt %, at least about 40 wt %, at least about 45 wt %, at least about 50 wt %, at least about 55 wt %, at least about 60 wt %, or at least about 70 wt % based on the total weight of the composition.

In a further aspect, the additive is present in an amount of 3 wt % or less based on the total weight of the composition. Thus, in various aspects, the additive is present in an amount of 2.5 wt % or less, 2 wt % or less, 1.5 wt % or less, 1 wt % or less, or 0.5 wt % or less based on the total weight of the composition.

In various aspects, the disclosed compositions can be in any form suitable for use such as, for example, a tablet, a capsule, lozenge, a troche, a suppository, a soft gel, a tincture, and the like. These formulations can be prepared via conventional processing methods known to one skilled in the art.

In various aspects, the disclosed compositions are formulated as an oral dosage form. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs, and solutions; while carriers such as starches, sugars, microcrystalline cellulose, diluents, granulating agents, lubricants, binders, disintegrating agents, and the like can be used to form oral solid preparations such as powders, capsules, and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In various aspects, one or more components of the disclosed compositions can be present as a pharmaceutically acceptable salt. In a further aspect, the pharmaceutically acceptable salt is a non-naturally occurring salt.

In various aspects the disclosed composition contains a pharmaceutically acceptable carrier. In a further aspect, the pharmaceutically acceptable carrier is a non-naturally occurring carrier. Examples of non-naturally occurring carriers include, but are not limited to, synthetic nanoparticles (e.g., based on fullerenes, nanotubes, or carbon black, in combination with a metal oxide, semiconductor, and/or metal), synthetic lipoproteins, semi-synthetic biopolymer complexes (e.g., polysaccharides complexed with synthetic polymers or synthetic chemicals), synthetic hydrogels (e.g., poly(hydroxyethyl methacrylate, poly(vinyl alcohol), poly(ethylene glycol), poly(acrylic acid), poly(methacrylic acid), polyacrylamide), and synthetic polymers (low density polyethylene, high density polyethylene, polypropylene, polyvinyl chloride, polystyrene, nylon, Teflon, thermoplastic polyurethane).

In various aspects, the disclosed composition is formulated for use in a device such as, for example, a vaporizer, a transdermal patch, or an inhaler.

It is understood that the disclosed compositions can be employed in the disclosed methods of using.

1. FLAVANOLS

In various aspects, the disclosed compositions comprise a flavanol. Flavanols are a group of bioactive compounds found in certain plant-based foods including teas, cocoa powder, chocolate, grapes, and blueberries. Additional sources of flavanols are known by those skilled in the art. In various aspects, flavanols can be extracted from, for example, fruits, vegetables, green tea, or other natural sources by any suitable method known by those of ordinary skill. Flavanols can be extracted from a single plant or from a mixture of plants. Examples of flavanols include, but are not limited to, kaempferol quercetin, myricetin, fisetin, and rutin. In a further aspect, the flavanol is selected from rutin and kaempferol. In a still further aspect, the flavanol is kaempferol.

The beneficial properties of flavanols are well-established. For example, cocoa flavanols have been shown to improve cerebral blood flow, synaptic plasticity, and mitochondrial function. Grape and grape juice (rich in catechin and epicatechin) appear capable of reducing glutamate excitotoxicity and exert powerful antioxidant activity. Thus, flavanols from these sources may ameliorate endothelial function, reduce platelet aggregation, and reduce low-density lipoprotein (LDL) oxidation. Additional benefits of flavanols are known by those of ordinary skill in the art.

In various aspects, inclusion of a flavanol, in combination with a cannabinol (CBN) in an amount of at least about 1 wt % based on the total weight of the composition, can result in significant improvements in the beneficial properties of the disclosed compositions. Exemplary beneficial properties include, but are not limited to, improvements in sleep quality, increased sleep duration, and decreased anxiety prior to sleep. Such improvements can be determined by, for example, providing a sample of the disclosed composition to a group of subjects, and then asking the subjects to provide feedback on their satisfaction with the composition. A similar study can be done with comparative compositions that do not contain both a cannabinol in an amount of at least about 1 wt % based on the total weight of the composition and a flavanol in order to evaluate and quantify the improvement in the beneficial properties. Thus, in various aspects, a composition that contains a cannabinoid and a flavanols can improve the beneficial properties (e.g., sleep quality, sleep duration, and/or anxiety) at least 1%, at least 5%, at least 10%, at least 15%, at least 20%, at least 25%, at least 30%, at least 35%, at least 40%, at least 45%, or at least 50% more effectively than the same composition in the absence of a flavanol. In various further aspects, a composition that contains a cannabinoid and a flavanols can treat a sleep disorder (e.g., by improving sleep quality, increasing sleep duration, and/or decreasing anxiety) at least 1%, at least 5%, at least 10%, at least 15%, at least 20%, at least 25%, at least 30%, at least 35%, at least 40%, at least 45%, or at least 50% more effectively than the same composition in the absence of a flavanols.

### C. METHODS FOR MAKING A COMPOSITION

In one aspect, disclosed are methods for making a disclosed composition. Thus, in various aspects, disclosed are methods for making a composition, the method comprising the step of combining: (a) cannabinol (CBN) in an amount of at least about 1 wt % based on the total weight of the composition; (b) one or more agents selected from a flavanol, an endocannabinoid, capsaicin, dihydrocapsaicin, nordihydrocapsaicin, homocapsaicin, and homodihydrocapsaicin; and (c) optionally, melatonin, wherein the composition is substantially free of surfactants.

In one aspect, disclosed are methods for making a composition, the method comprising the step of combining: (a) CBN in an amount of at least about 1 wt % based on the total weight of the composition; and (b) melatonin, wherein the composition is substantially free of surfactants.

In one aspect, disclosed are methods for making a composition, the method comprising the step of combining: (a) CBN in an amount of at least about 1 wt % based on the total weight of the composition; and (b) melatonin in an amount of from about 0.5 wt % to about 5 wt % based on the total weight of the composition, wherein the ratio of CBN to melatonin is of from about 1:1 by weight to about 5:1 by weight and wherein the composition is substantially free of surfactants.

Thus, in various aspects, CBN is present in an amount of at least about 1 wt %, at least about 2 wt %, at least about 5 wt %, at least about 10 wt %, at least about 15 wt %, at least about 20 wt % at least about 25 wt %, at least about 30 wt %, at least about 35 wt %, or at least about 40 wt % based on the total weight of the composition.

In various aspects, the one or more agents selected from a flavanol, an endocannabinoid, capsaicin, dihydrocapsaicin, nordihydrocapsaicin, homocapsaicin, and homodihydrocapsaicin is present. In a further aspect, a flavanol is present. In a still further aspect, an endocannabinoid is present. In yet a further aspect, one or more of capsaicin, dihydrocapsaicin, nordihydrocapsaicin, homocapsaicin, and homodihydrocapsaicin is present.

In various aspects, one or more agents is present in an amount of from about 0.5 wt % to about 30 wt %, about 0.5 wt % to about 25 wt %, about 0.5 wt % to about 20 wt %, about 0.5 wt % to about 15 wt %, about 0.5 wt % to about 10 wt %, about 0.5 wt % to about 8 wt %, about 0.5 wt % to about 6 wt %, about 0.5 wt % to about 4 wt %, about 0.5 wt % to about 2 wt %, about 0.5 wt % to about 1 wt %, about 0.5 wt % to about 30 wt %, about 1 wt % to about 30 wt %, about 2 wt % to about 30 wt %, about 4 wt % to about 30 wt %, about 6 wt % to about 30 wt %, about 8 wt % to about 30 wt %, about 10 wt % to about 30 wt %, about 15 wt % to about 30 wt %, about 20 wt % to about 30 wt %, about 25 wt % to about 30 wt %, about 1 wt % to about 25 wt %, about 2 wt % to about 20 wt %, about 4 wt % to about 20 wt %, about 6 wt % to about 15 wt %, or about 8 wt % to about 10 wt %, based on the total weight of the composition.

In various aspects, the flavanol is present. Examples of flavanols include, but are not limited to, kaempferol and rutin. In a further aspect, the flavanol is kaempferol. Thus, the flavanol can be present in an amount of from about 0.5 wt % to about 30 wt %, about 0.5 wt % to about 25 wt %, about 0.5 wt % to about 20 wt %, about 0.5 wt % to about 15 wt %, about 0.5 wt % to about 10 wt %, about 0.5 wt % to about 8 wt %, about 0.5 wt % to about 6 wt %, about 0.5 wt % to about 4 wt %, about 0.5 wt % to about 2 wt %, about 0.5 wt % to about 1 wt %, about 0.5 wt % to about 30 wt %, about 1 wt % to about 30 wt %, about 2 wt % to about 30 wt %, about 4 wt % to about 30 wt %, about 6 wt % to about 30 wt %, about 8 wt % to about 30 wt %, about 10 wt % to about 30 wt %, about 15 wt % to about 30 wt %, about 20 wt % to about 30 wt %, about 25 wt % to about 30 wt %, about 1 wt % to about 25 wt %, about 2 wt % to about 20 wt %, about 4 wt % to about 20 wt %, about 6 wt % to about 15 wt %, or about 8 wt % to about 10 wt %, based on the total weight of the composition.

In various aspects, the composition consists essentially of CBN, the flavanol, and melatonin. In a further aspect, the composition consists of CBN, the flavanol, and melatonin.

In various aspects, the endocannabinoid is present. Examples of endocannabinoids include, but are not limited to, 2-arachidonoylglycerol (2-AG), 2-arachidonyl glyceryl ether (2-AGE), N-arachidonoyl dopamine (NADA), stearoylethanolamide (SEA), O-arachidonoyl ethanolamine (O-AEA), arachidonoyl ethanolamide (AEA), 2-oleoylglycerol (2-OG), N-arachidonylglycine (NAGly), 2-arachidonoyl lysophosphatidylinositol (2-ALPI), N-arachidonoyl serotonin (AA-5-HT), docosatetraenoylethanolamide (DEA), lysophosphatidylinositol (LPI), oleamide, oleoylethaolamide (OEA), palmitoylethanolamide (PEA), RVD-Hpa, stearoylethanolamide (SEA), yangonin, and an alkylamide (e.g., Echinacea and its constituents). Thus, the endocannabinoid can be present in an amount of from about 0.5 wt % to about 30 wt %, about 0.5 wt % to about 25 wt %, about 0.5 wt % to about 20 wt %, about 0.5 wt % to about 15 wt %, about 0.5 wt % to about 10 wt %, about 0.5 wt % to about 8 wt %, about 0.5 wt % to about 6 wt %, about 0.5 wt % to about 4 wt %, about 0.5 wt % to about 2 wt %, about 0.5 wt % to about 1 wt %, about 0.5 wt % to about 30 wt %, about 1 wt % to about 30 wt %, about 2 wt % to about 30 wt %, about 4 wt % to about 30 wt %, about 6 wt % to about 30 wt %, about 8 wt % to about 30 wt %, about 10 wt % to about 30 wt %, about 15 wt % to about 30 wt %, about 20 wt % to about 30 wt %, about 25 wt % to about 30 wt %, about 1 wt % to about 25 wt %, about 2 wt % to about 20 wt %, about 4 wt % to about 20 wt %, about 6 wt % to about 15 wt %, or about 8 wt % to about 10 wt %, based on the total weight of the composition.

In various aspects, the composition consists essentially of CBN, the endocannabinoid, and melatonin. In a further aspect, the composition consists of CBN, the endocannabinoid, and melatonin.

In various aspects, melatonin is present in an amount of from about 0.5 wt % to about 5 wt %, about 0.5 wt % to about 4 wt %, about 0.5 wt % to about 3 wt %, about 0.5 wt % to about 2 wt %, about 0.5 wt % to about 1 wt %, about 1 wt % to about 5 wt %, about 2 wt % to about 5 wt %, about 3 wt % to about 5 wt %, about 4 wt % to about 5 wt %, about 1 wt % to about 4 wt % or about 2 wt % to about 3 wt % based on the total weight of the composition.

In various aspects, the ratio of CBN to melatonin is of from about 1:1 by weight to about 5:1 by weight, from about 1:1 by weight to about 4:1 by weight, from about 1:1 by weight to about 3:1 by weight, from about 1:1 by weight to about 2:1 by weight, from about 2:1 by weight to about 5:1 by weight, from about 3:1 by weight to about 5:1 by weight, from about 4:1 by weight to about 5:1 by weight, or from about 2:1 by weight to about 4:1 by weight.

In various aspects, the composition consists essentially of CBN and melatonin. In a further aspect, the composition consists of CBN and melatonin.

In various aspects, the composition is substantially free of surfactants. Thus, in various further aspects, the composition contains less than about 10% by weight, less than about 5%, less than about 1%, less than about 0.5%, less than about 0.1%, less than about 0.05%, or less than about 0.01% by weight of surfactants based on the total weight of the composition.

In a further aspect, the composition further comprises an oil. Examples of oils include, but are not limited to, tocopheryl acetate, olive oil, almond oil, apricot kernel oil, avocado oil, borage seed oil, camellia seed oil (tea oil), cranberry seed oil, evening primrose oil, grapeseed oil, hazelnut oil, hemp seed oil, jojoba, kukui nut oil, macademia nut oil, meadowfoam oil, peanut oil, pecan oil, pomegranate seed oil, rose hip oil, seabuckthorn berry oil, sesame seed oil, coconut oil, sunflower oil, watermelon seed oil, and MCT oil, or mixtures thereof In a still further aspect, the oil is tocopheryl acetate.

Without wishing to be bound by theory, the amount of oil present can be dependent upon the formulation of the composition. For example, in various aspects, the oil is present in an amount of from about 20 wt % to about 30 wt %, 25 wt % to about 30 wt %, or 20 wt % to about 25 wt %. In various further aspects, the oil is present in an amount of from about 50 wt % to about 98 wt %, 60 wt % to about 98 wt %, 70 wt % to about 98 wt %, 80 wt % to about 98 wt %, 90 wt % to about 98 wt %, 50 wt % to about 90 wt %, 50 wt % to about 80 wt %, 50 wt % to about 70 wt %, or 50 wt % to about 60 wt %. In a further aspect, the oil is present in an amount of from about 50 wt % to about 98 wt % based on the total weight of the composition.

In various aspects, the oil is naturally occurring. In a still further aspect, the oil is non-naturally occurring (e.g., MCT oil).

In various aspects, the composition further comprises a cannabinoid selected from Δ9-tetrahydrocannabinol (THC), cannabidiol (CBD), cannabigerol (CBG), cannabicyclol (CBL), and cannabichromene (CBC), or a mixture thereof. In a further aspect, the cannabinoid is present in an amount of from about 1 wt % to about 20 wt % based on the total weight of the composition. Thus, in various aspects, the cannabinoid is present in an amount of from about 1 wt % to about 15 wt %, about 1 wt % to about 10 wt %, about 1 wt % to about 5 wt %, about 5 wt % to about 20 wt %, about 10 wt % to about 20 wt %, or about 15 wt % to about 20 wt % based on the total weight of the composition.

In various aspects, the composition further comprises a cannabinoid acid. Examples of cannabinoid acids include, but are not limited to, CBGA, THCA, CBDA, CBCA, CBGVA, THCVA, CBDVA, and CBCVA. In a further aspect, the cannabinoid acid is present in an amount of from about 1 wt % to about 15 wt %, about 1 wt % to about 10 wt %, about 1 wt % to about 5 wt %, about 5 wt % to about 20 wt %, about 10 wt % to about 20 wt %, or about 15 wt % to about 20 wt % based on the total weight of the composition.

In various aspects, the composition is substantially free of THC. Thus, in various further aspects, the composition contains less than about 10% by weight, less than about 5%, less than about 1%, less than about 0.5%, less than about 0.1%, less than about 0.05%, or less than about 0.01% by weight of THC based on the total weight of the composition.

In various aspects, the composition is substantially free of CBN. Thus, in various further aspects, the composition contains less than about 10% by weight, less than about 5%, less than about 1%, less than about 0.5%, less than about 0.1%, less than about 0.05%, or less than about 0.01% by weight of CBN based on the total weight of the composition.

In various aspects, the composition is substantially free of THC and CBN. Thus, in various further aspects, the composition contains less than about 10% by weight, less than about 5%, less than about 1%, less than about 0.5%, less than about 0.1%, less than about 0.05%, or less than about 0.01% by weight of both THC and CBN based on the total weight of the composition.

In various aspects, the composition further comprises an additive. Examples of additives include, but are not limited to, diluents, buffers, binders, surface-active agents, lubricants, humectants, pH adjusting agents, preservatives (including anti-oxidants), emulsifiers, occlusive agents, opacifiers, antioxidants, colorants, flavoring agents, gelling agents, thickening agents, stabilizers, and surfactants, among others. Thus, in various further aspects, the additive is vitamin E, gum acacia, citric acid, stevia extract powder, Luo Han Gou, Monoammonium Glycyrhizinate, Ammonium Glycyrrhizinate, honey, or combinations thereof. In a still further aspect, the additive is a flavoring agent, a binder, a disintegrant, a bulking agent, and/or silica.

In a further aspect, the additive is present in an amount of from about 0.01 wt % to about 10 wt % based on the total weight of the composition. In a still further aspect, the additive is present in an amount of from about 0.01 wt % to about 8 wt %, from about 0.01 wt % to about 6 wt %, from about 0.01 wt % to about 4 wt %, from about 0.01 wt % to about 2 wt %, from about 0.01 wt % to about 1 wt %, from about 0.1 wt % to about 10 wt %, from about 1 wt % to about 10 wt %, from about 2 wt % to about 10 wt %, from about 4 wt % to about 10 wt %, from about 6 wt % to about 10 wt %, or from about 8 wt % to about 10 wt % based on the total weight of the composition.

In a further aspect, the additive is present in an amount of at least about 20 wt % based on the total weight of the composition. Thus, in various aspects, the additive is present in an amount of at least about 20 wt %, at least about 25 wt %, at least about 30 wt %, at least about 35 wt %, at least about 40 wt %, at least about 45 wt %, at least about 50 wt %, at least about 55 wt %, at least about 60 wt %, or at least about 70 wt % based on the total weight of the composition.

In a further aspect, the additive is present in an amount of 3 wt % or less based on the total weight of the composition. Thus, in various aspects, the additive is present in an amount of 2.5 wt % or less, 2 wt % or less, 1.5 wt % or less, 1 wt % or less, or 0.5 wt % or less based on the total weight of the composition.

### D. METHODS OF USING THE COMPOSITIONS

The disclosed compositions are useful in inducing or promoting sleep. The disclosed compositions are also useful in treating sleep disorders such as, for example, insomnia, SAD, and jet lag.

The compositions can be administered to the subject via, for example, oral administration (e.g., as a tablet, capsule, lozenge, or troche). Alternatively, the compositions can be administered via a device such as, for example, a vaporizer, transdermal patch, or inhaler. Administration can be continuous or intermittent. A preparation can be administered therapeutically; that is, administered to treat or ameliorate an existing disorder or condition.

The therapeutically effective amount or dosage of the composition can vary within wide limits. Such a dosage is adjusted to the individual requirements in each particular case including the specific composition(s) being administered and the condition being treated, as well as the patient being treated. In general, single dose compositions can contain such amounts or submultiples thereof of the composition to make up the daily dose. The dosage can be adjusted by the individual physician in the event of any contraindications. Dosage can vary, and can be administered in one or more dose administrations daily, for one or several days.

In a further aspect, the subject is a mammal. In a still further aspect, the mammal is a human.

In a further aspect, administering is via a vaporizer, a transdermal patch, or an inhaler.

1. TREATMENT METHODS

The compounds disclosed herein are useful for inducing or promoting sleep, as well as for treating sleep disorders such as, for example, insomnia, SAD, and jet lag. Thus, provided is a method comprising administering a therapeutically effective amount of a disclosed composition to a subject. In a further aspect, the method can be a method for treating a sleep disorder. In a still further aspect, the method can be a method for promoting sleep.

a. TREATING A SLEEP DISORDER

In one aspect, disclosed are methods for treating a sleep disorder in a subject having the sleep disorder, the method comprising administering to the subject a therapeutically effective amount of a disclosed composition. Examples of sleep disorders include, but are not limited to, insomnia, SAD, and jet lag.

Thus, in one aspect, disclosed are methods for treating a sleep disorder in a subject having the sleep disorder, the method comprising administering to the subject a therapeutically effective amount of a composition comprising: (a) cannabinol (CBN) in an amount of at least about 1 wt % based on the total weight of the composition; (b) one or more agents selected from a flavanol, an endocannabinoid, capsaicin, dihydrocapsaicin, nordihydrocapsaicin, homocapsaicin, and homodihydrocapsaicin; and (c) optionally, melatonin, wherein the composition is substantially free of surfactants.

In one aspect, disclosed are methods for treating a sleep disorder in a subject having the sleep disorder, the method comprising administering to the subject a therapeutically effective amount of a composition comprising: (a) CBN in an amount of at least about 1 wt % based on the total weight of the composition; and (b) melatonin, wherein the composition is substantially free of surfactants.

In one aspect, disclosed are methods for treating a sleep disorder in a subject having the sleep disorder, the method comprising administering to the subject a therapeutically effective amount of a composition comprising: (a) CBN in an amount of at least about 1 wt % based on the total weight of the composition; and (b) melatonin in an amount of from about 0.5 wt % to about 5 wt % based on the total weight of the composition, wherein the ratio of CBN to melatonin is of from about 1:1 by weight to about 5:1 by weight and wherein the composition is substantially free of surfactants.

In various aspects, the subject is a mammal. In a further aspect, the subject is a human.

In a further aspect, the method further comprises the step of identifying a subject in need of treatment of the disorder. In a still further aspect, the subject has been diagnosed with a need for treatment of the disorder prior to the administering step.

In a further aspect, the method further comprises administering at least one agent known for the treatment of sleep disorders, wherein the composition and the agent are not co-formulated. Examples of agents known for the treatment of sleep disorders include, but are not limited to, melatonin, valerian, chamomile, kava kava, antihistamines, and sedatives.

In a further aspect, the agent is an antihistamine. Examples of antihistamines include, but are not limited to, diphenhydramine and doxylamine.

In a further aspect, the agent is a sedative. Examples of sedatives include, but are not limited to, short-acting sedative-hypnotics (e.g., zolpidem, zaleplon, eszopiclone), orexin receptor antagonists (e.g., suvorexant), melatonin receptor agonists (e.g., ramelteon), benzodiazepines (e.g., flurazepan, temazepam, estazolam), and tricyclic antidepressants (e.g., doxepin).

In a further aspect, the agent and the composition are administered sequentially. In a still further aspect, the agent and the composition are administered simultaneously.

b. PROMOTING SLEEP

In one aspect, disclosed are methods for inducing and/or promoting sleep in a subject, the method comprising administering to the subject a therapeutically effective amount of a disclosed composition. Thus, in one aspect, disclosed are methods for inducing and/or promoting sleep in a subject, the method comprising administering to the subject a therapeutically effective amount of a composition comprising: (a) cannabinol (CBN) in an amount of at least about 1 wt % based on the total weight of the composition; (b) one or more agents selected from a flavanol, an endocannabinoid, capsaicin, dihydrocapsaicin, nordihydrocapsaicin, homocapsaicin, and homodihydrocapsaicin; and (c) optionally, melatonin, wherein the composition is substantially free of surfactants. In a further aspect, disclosed are methods for inducing sleep in a subject. In a still further aspect, disclosed are methods for promoting sleep in a subject.

In one aspect, disclosed are methods for inducing and/or promoting sleep in a subject, the method comprising administering to the subject a therapeutically effective amount of a composition comprising: (a) CBN in an amount of at least about 1 wt % based on the total weight of the composition; and (b) melatonin, wherein the composition is substantially free of surfactants.

In one aspect, disclosed are methods for inducing and/or promoting sleep in a subject, the method comprising administering to the subject a therapeutically effective amount of a composition comprising: (a) CBN in an amount of at least about 1 wt % based on the total weight of the composition; and (b) melatonin in an amount of from about 0.5 wt % to about 5 wt % based on the total weight of the composition, wherein the ratio of CBN to melatonin is of from about 1:1 by weight to about 5:1 by weight and wherein the composition is substantially free of surfactants.

In various aspects, the subject is a mammal. In a further aspect, the subject is a human.

In a further aspect, the method further comprises the step of identifying a subject in need of assistance in falling and/or staying asleep. In a still further aspect, the subject has been diagnosed with a need for assistance in falling and/or staying asleep.

In a further aspect, the method further comprises administering at least one agent known for inducing and/or promoting sleep, wherein the composition and the agent are not co-formulated. Examples of agents known for the treatment of sleep disorders include, but are not limited to, melatonin, valerian, chamomile, kava kava, antihistamines, and sedatives.

In a further aspect, the agent is an antihistamine. Examples of antihistamines include, but are not limited to, diphenhydramine and doxylamine.

In a further aspect, the agent is a sedative. Examples of sedatives include, but are not limited to, short-acting sedative-hypnotics (e.g., zolpidem, zaleplon, eszopiclone), orexin receptor antagonists (e.g., suvorexant), melatonin receptor agonists (e.g., ramelteon), benzodiazepines (e.g., flurazepan, temazepam, estazolam), and tricyclic antidepressants (e.g., doxepin).

In a further aspect, the agent and the composition are administered sequentially. In a still further aspect, the agent and the composition are administered simultaneously.

2. USE OF COMPOSITIONS

In one aspect, the invention relates to the use of a disclosed composition or a product of a disclosed method. In a further aspect, a use relates to the manufacture of a medicament for the treatment of a sleep disorder in a subject. In a still further aspect, a use relates to the manufacture of a medicament for inducing and/or promoting sleep in a subject.

Also provided are the uses of the disclosed compositions and products. In one aspect, the invention relates to use of at least one disclosed composition. In a further aspect, the composition used is a product of a disclosed method of making.

In a further aspect, the use relates to a process for preparing a pharmaceutical composition comprising a therapeutically effective amount of a disclosed composition or a product of a disclosed method of making, for use as a medicament.

In a further aspect, the use relates to a process for preparing a pharmaceutical composition comprising a therapeutically effective amount of a disclosed composition or a product of a disclosed method of making, wherein a pharmaceutically acceptable carrier is intimately mixed with a therapeutically effective amount of the composition or the product of a disclosed method of making.

In various aspects, the use relates to a treatment of a sleep disorder a subject. In one aspect, the use is characterized in that the subject is a human. In one aspect, the use is characterized in that the sleep disorder is insomnia, SAD, or jet lag.

In various aspects, the use relates to inducing and/or promoting sleep in a subject. Thus, in a further aspect, the use relates to inducing sleep in a subject. In a still further aspect, the use relates to promoting sleep in a subject. In one aspect, the use is characterized in that the subject is a human.

In a further aspect, the use relates to the manufacture of a medicament for the treatment of a sleep disorder in a subject.

In a further aspect, the use relates to the manufacture of a medicament for inducing and/or promoting sleep in a subject. In a still further aspect, the use relates to the manufacture of a medicament for inducing sleep in a subject. In yet a further aspect, the use relates to the manufacture of a medicament for promoting sleep in a subject.

It is understood that the disclosed uses can be employed in connection with the disclosed compositions, products of disclosed methods of making, methods, and kits. In a further aspect, the invention relates to the use of a disclosed composition or a disclosed product in the manufacture of a medicament for inducing and/or promoting sleep in a mammal. In a still further aspect, the invention relates to the use of a disclosed composition or a disclosed product in the manufacture of a medicament for the treatment of a sleep disorder in a mammal. In a further aspect, the sleep disorder is insomnia, SAD, or jet lag.

3. MANUFACTURE OF A MEDICAMENT

In one aspect, the invention relates to a method for the manufacture of a medicament for treating a sleep disorder in a subject, the method comprising combining a therapeutically effective amount of a disclosed composition or product of a disclosed method with a pharmaceutically acceptable carrier or diluent.

In one aspect, the invention relates to a method for the manufacture of a medicament for inducing and/or promoting in a subject, the method comprising combining a therapeutically effective amount of a disclosed composition or product of a disclosed method with a pharmaceutically acceptable carrier or diluent.

As regards these applications, the present method includes the administration to an animal, particularly a mammal, and more particularly a human, of a therapeutically effective amount of the composition effective in the treatment of the disorder. The dose administered to an animal, particularly a human, in the context of the present invention should be sufficient to affect a therapeutic response in the animal over a reasonable time frame. One skilled in the art will recognize that dosage will depend upon a variety of factors including the condition of the animal and the body weight of the animal.

The size of the dose also will be determined by the route, timing, and frequency of administration as well as the existence, nature, and extent of any adverse side effects that might accompany the administration of the composition and the desired physiological effect. It will be appreciated by one of skill in the art that various conditions or disorders, in particular chronic conditions or disorders, may require prolonged treatment involving multiple administrations.

Thus, in one aspect, the invention relates to the manufacture of a medicament comprising combining a disclosed composition or a product of a disclosed method of making, with a pharmaceutically acceptable carrier or diluent.

### E. KITS

In one aspect, disclosed are kits comprising a disclosed composition. Thus, in various aspects, disclosed are kits comprising a composition and one or more of: (a) a device selected from a vaporizer, a transdermal patch, or an inhaler; (b) an agent known to promote sleep and/or to treat a sleep disorder; (c) instructions for promoting sleep; and (d) instructions for treating a sleep disorder, wherein the composition comprises: (a) cannabinol (CBN) in an amount of at least about 1 wt % based on the total weight of the composition; (b) one or more agents selected from a flavanol, an endocannabinoid, capsaicin, dihydrocapsaicin, nordihydrocapsaicin, homocapsaicin, and homodihydrocapsaicin; and (c) optionally, melatonin, wherein the composition is substantially free of surfactants.

In one aspect, disclosed are kits comprising a composition and one or more of: (a) a device selected from a vaporizer, a transdermal patch, or an inhaler; (b) an agent known to promote sleep and/or to treat a sleep disorder; (c) instructions for promoting sleep; and (d) instructions for treating a sleep disorder, wherein the composition comprises: (e) CBN in an amount of at least about 1 wt % based on the total weight of the composition; and (f) melatonin, and wherein the composition is substantially free of surfactants.

In one aspect, disclosed are kits comprising a composition and one or more of: (a) a device selected from a vaporizer, a transdermal patch, or an inhaler; (b) an agent known to promote sleep and/or to treat a sleep disorder; (c) instructions for promoting sleep; and (d) instructions for treating a sleep disorder, wherein the composition comprises: (e) CBN in an amount of at least about 1 wt % based on the total weight of the composition; and (f) melatonin in an amount of from about 0.5 wt % to about 5 wt % based on the total weight of the composition, wherein the ratio of CBN to melatonin is of from about 1:1 by weight to about 5:1 by weight, and wherein the composition is substantially free of surfactants.

In a further aspect, the composition and the agent known to promote sleep and/or to treat a sleep disorder are co-packaged. In a still further aspect, the composition and the agent known to promote sleep and/or to treat a sleep disorder are not co-packaged.

In a further aspect, the kit comprises the composition and the agent known to promote sleep and/or to treat a sleep disorder. Examples of agents known for the treatment of sleep disorders include, but are not limited to, melatonin, valerian, chamomile, kava kava, antihistamines, and sedatives.

In a further aspect, the agent known to promote sleep and/or to treat a sleep disorder is an antihistamine. Examples of antihistamines include, but are not limited to, diphenhydramine and doxylamine.

In a further aspect, the agent known to promote sleep and/or to treat a sleep disorder is a sedative. Examples of sedatives include, but are not limited to, short-acting sedative-hypnotics (e.g., zolpidem, zaleplon, eszopiclone), orexin receptor antagonists (e.g., suvorexant), melatonin receptor agonists (e.g., ramelteon), benzodiazepines (e.g., flurazepan, temazepam, estazolam), and tricyclic antidepressants (e.g., doxepin).

In a further aspect, the agent known to promote sleep and/or to treat a sleep disorder and the composition are administered sequentially. In a still further aspect, the agent known to promote sleep and/or to treat a sleep disorder and the composition are administered simultaneously.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The previous description of the disclosed aspects is provided to enable any person skilled in the art to make or use the present disclosure. Various modifications to these aspects will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method for promoting sleep in a subject, the method comprising administering to the subject an effective amount of a composition comprising:
(a) cannabinol (CBN) in an amount of at least about 1 wt % based on the total weight of the composition;
(b) one or more agents selected from a flavanol, an endocannabinoid, capsaicin, dihydrocapsaicin, nordihydrocapsaicin, homocapsaicin, and homodihydrocapsaicin; and
(c) optionally, melatonin, wherein the composition is substantially free of surfactants.

2. The method of claim 1, wherein administering is via a vaporizer, a transdermal patch, or an inhaler.

3. A method for treating a sleep disorder in a subject having the sleep disorder, the method comprising administering to the subject a therapeutically effective amount of a composition comprising:
(a) cannabinol (CBN) in an amount of at least about 1 wt % based on the total weight of the composition;
(b) one or more agents selected from a flavanol, an endocannabinoid, capsaicin, dihydrocapsaicin, nordihydrocapsaicin, homocapsaicin, and homodihydrocapsaicin; and
(c) optionally, melatonin, wherein the composition is substantially free of surfactants.

4. The method of claim 3, wherein the sleep disorder is insomnia, seasonal affective disorder (SAD), or jet lag.

5. A composition comprising:
(a) cannabinol (CBN) in an amount of at least about 1 wt % based on the total weight of the composition;
(b) one or more agents selected from a flavanol, an endocannabinoid, capsaicin, dihydrocapsaicin, nordihydrocapsaicin, homocapsaicin, and homodihydrocapsaicin; and
(c) optionally, melatonin, wherein the composition is substantially free of surfactants.

6. The composition of claim 5, wherein the flavanol is present.

7. The composition of claim 6, wherein the favanol is selected from kaempferol and rutin.

8. The composition of claim 5, wherein the endocannabinoid is present.

9. The composition of claim 8, wherein the endocannabinoid is selected from 2-arachidonoylglycerol (2-AG), 2-arachidonyl glyceryl ether (2-AGE), N-arachidonoyl dopamine (NADA), stearoylethanolamide (SEA), O-arachidonoyl ethanolamine (O-AEA), arachidonoyl ethanolamide (AEA), 2-oleoylglycerol (2-OG), N-arachidonylglycine (NAGly), 2-arachidonoyl lysophosphatidylinositol (2-ALPI), N-arachidonoyl serotonin (AA-5-HT), docosatetraenoylethanolamide (DEA), lysophosphatidylinositol (LPI), oleamide, oleoylethaolamide (OEA), palmitoylethanolamide (PEA), RVD-Hpa, stearoylethanolamide (SEA), yangonin, and an alkylamide (e.g., Echinacea and its constituents).

10. The composition of claim 5, wherein the agent is present in an amount of from about 0.5 wt % to about 10 wt %.

11. The composition of claim 5, wherein melatonin is present.

12. The composition of claim 11, wherein the melatonin is present in an amount of from about 0.5 wt % to about 5 wt % based on the total weight of the composition.

13. The composition of claim 11, wherein the ratio of CBN to melatonin is of from about 1:1 by weight to about 5:1 by weight.

14. The composition of claim 11, wherein the composition consists essentially of CBN, the flavanol, and melatonin.

15. The composition of claim 5, further comprising an oil.

16. The composition of claim 5, further comprising a cannabinoid selected from Δ9-tetrahydrocannabinol (THC), cannabidiol (CBD), cannabigerol (CBG), cannabicyclol (CBL), and cannabichromene (CBC), or a mixture thereof.

17. The composition of claim 5, further comprising a cannabinoid acid.

18. The composition of claim 5, wherein the composition is substantially free of THC and CBD.

19. The composition of claim 5, formulated as a tablet, a capsule, lozenge, a troche, a suppository, a soft gel, or a tincture.

20. The composition of claim 5, formulated for use in a vaporizer, a transdermal patch, or an inhaler.
